(19) United States
(12) Patent Application Publication
(10) Pub. No.: US 2012/0215148 A1
(43) Pub. Date: Aug. 23, 2012

(54) HIGHLY FUNCTIONAL SPUNBONDED FABRIC MADE FROM PARTICLE-CONTAINING FIBRES AND METHOD FOR PRODUCING SAME

(76) Inventors: Yvonne Ewert, Rudolstadt (DE); Frank-Günter Niemz, Rudolstadt (DE); Marcus Krieg, Weimar (DE); Bernd Riedel, Unterwellenborn (DE)

(21) Appl. No.: 13/504,567

(22) PCT Filed: Sep. 13, 2011
(86) PCT No.: PCT/EP2011/004591

§ 371 Date: Apr. 27, 2012

(30) Foreign Application Priority Data
Sep. 14, 2010 (DE) 10 2010 045 242.4

(51) Int. Cl. A61F 13/00; D04H 3/11; A41D 27/02; D04H 3/16; D01F 6/00; D04H 3/105; D04H 3/12

(52) U.S. Cl. 602/45; 442/336; 428/219; 428/220; 264/115; 264/103; 2/272

(57) ABSTRACT

The invention relates to functional spunbonded fabrics incorporating fibers made from non-fusible polymers containing one or more functional additives. The fibers are interwoven and interlocked to form a firm fleece composite, have different lengths, and have aspect ratios above 1,000. The fibers have a mean diameter of 0.1 to 500 micrometres and diameter variations within a fiber and/or among each other of at least 30%. The fibers contain more than 40 wt % of finely distributed functional additives in solid and/or liquid form. The spunbonded fabric is produced from a spinning solution containing the non-fusible polymer dissolved in a direct solvent and at least one functional additive. The spinning solution is extruded out of a spinneret, and the resulting strands are drawn in the longitudinal direction to form filaments or fibers, stabilized and laid down to form a fleece fabric. Exemplary spunbonded fabrics include clothing, technical textiles and filters.

The invention relates to high-functionality spunbondeds as textile fabrics which are obtainable directly from dissolved polymers using known spunbond processes and which are constructed of fibers filled with liquid and/or solid functional additives. The fibers consist of functional additive to an extent of more than 40 wt %, based on total fiber weight, the average fiber diameter is in the range from 0.1 to 500 micrometers, and diameter fluctuations within and between fibers amount to at least 30%. The spunbonded has high functionality due to very high concentrations of functional additives and is versatile in use, for example as interlining, for hygiene applications, for wound dressings, as carrier materials, as building and transportation material, as cosmetic material or as a filter.

Filled textile fabrics, for example those having thermoregulating, antimicrobial or absorbent properties, are already known.

Prior art materials used in the form of textile fabrics require at least one additional processing step after the production of functional fibers, and/or contain only small amounts of functional particles. DE 10 2008 045290 A1 for example discloses fibers which are then used to produce textiles, wound dressings, filters, etc. Additive fractions are exclusively limited to zinc white (ZnO and ZnS), the fraction of which is limited to a maximum of 30%, while particle sizes are less than 15 μm. It is mentioned that the particle content can also be higher for fibrous nonwoven web applications, but no teaching is communicated as to how such nonwoven fabrics are obtainable. The object was not a functional fibrous nonwoven web having a high particle content, but washable and dyeable bactericidal moldings/fibers combining controlled delivery of active ingredients with prescribed durability to washing.

Carrier material often utilizes thermoplastically processable polymers whose melts can be processed into a spunbonded, for example in EP 1 199 393 A2. A spunbonded produced from thermoplastic polymers with hydrophobic admixtures is concerned there. The purpose is to concentrate the hydrophobic admixtures at the fiber surface. This is achieved by the fiber being by the air stream to such an extent that the average fiber diameter is equal to the particle diameter or decreases up to at most half the particle diameter. The fraction of masterbatches with the mixture agents is between 10 and 20 wt % and must not be higher so as not to impair the further processing into roofing membranes or the use in sanitary napkins.

Particle-containing filaments/fibers are not consistently obtainable in normal filament/fiber-spinning processes when the fraction of functional particles is very high at not less than 40%, since broken ends would be a frequent consequence.

Various sectors of the textile industry have a high need for fiber materials with additional functional benefit for the consumer, which shall also be inexpensive to produce and easy to process. Application sectors for such fiber materials include for example as interlining material in the apparel industry, industrial textiles, for example hygiene applications, wound dressings, as carrier materials, as building and transportation material, as cosmetic material or as filters, for example for the filtration of wastewater or exit air and binding of air and water ingredients.

Fabrics comprising functional additives are obtainable in principle either by fabric production along a textile value-added chain or fibrous nonwoven web formation in each case from functionally additized fibers, the coating of sheetlike textile structures with additive dispersions or the incorporation of solid or liquid functional additives in already produced fibrous nonwoven web structures.

Fibers having a fraction of functional additives which amounts to more than 40 wt % are not consistently obtainable in normal fiber-spinning processes, since broken fibers are a frequent consequence. Although this disadvantage can be partly redeemed in the use of functional fibers fabricated via solution spinning, the subsequent textile fabric production processes also always require at least one additional processing step.

Prior art materials produced in the form of functional textile fabrics require at least one additional processing step following a separate production of functional fibers, and/or contain only small amounts of functional particles. The separate operation to produce a fibrous nonwoven web imposes an additional stress on the highly filled fibers and as a result these are damaged and only meet comparatively low-quality requirements in respect of functionality and/or mechanical durability.

The present invention has for its purpose to provide a versatile fabric comprising particle-containing filaments and fibers with high functional benefit for various use sectors, depending on the nature of the functional particles, wherein the particle-containing filaments/fibers consist of functional additives to an extent of more than 40 wt % and have an average diameter in the range from 0.1 to 500 micrometers. The fabrics shall be sufficiently strong as-laid that they are suitable for further processing and/or direct use. Owing to the high fractions of functional admixtures, these fabrics shall have such functional properties as are otherwise only achievable through additional process steps such as coating or surface finishing.

These objects are achieved according to the present invention when directly in the spinning process a high-functionality textile fabric is produced continuously from a solution of nonmeltable polymers in direct solvents which is doped with one or more functional admixture agents, using a spunbond process. Surprisingly, textile fabrics having an additive content of more than wt % are consistently and reproducibly obtainable without additional process steps and retain permanent functionality throughout their entire life cycle. It was similarly found that the fibrous nonwoven web fabric fibers of the present invention have diameter fluctuations of at least 30% within and between fibers and, as a surprising result, have a high self-binding capacity through entangling and intertwining.

The present invention accordingly provides a high-functionality spunbonded web of fibers based on nonmeltable polymers containing one or more functional additives, characterized in that the fibers are intertwined and entangled, have a differing length with aspect ratios above 1000 and form a firmly interbonded web, wherein the fibers have an average diameter of 0.1 to 500 micrometers and also diameter fluctuations of at least 30% within any one fiber and/or between fibers and wherein the fibers in addition to the nonmeltable polymer contain more than 40 wt %, based on total fiber weight, of functional additives in solid and/or liquid form which are finely distributed in the fibers.

The textile fabrics which are useful for various application sectors depending on the type of functional additives are constructed from additive-containing fibers containing more than 40 wt % and up to 96 wt %, optionally even more, based on total fiber weight in each case, of functional additive and have an average diameter from 0.1 to 500 micrometers. The fraction of functional additives is preferably more than 40 wt % up to 90 wt %, based on total fiber weight.

The incorporated and permanent functions of the additives range for example from electrically conductive, absorbing, ion exchanging, antibacterial, temperature regulating through to flame retardant, abrasive or reconditioning, and/or combinations thereof.

The functional additives are in particular activated carbon, superabsorbents, ion exchange resins, PCM, metal oxides, flame retardants, abrasives, zeolites, sheet-silicates, such as bentonites, or modified sheet-silicates, cosmetics or mixtures thereof. Liquid lipophilic substances, such as paraffins, waxes or oils, can also be introduced as functional additive. In addition, one or further components can be introduced in minor concentrations, for example nanosilver or dyes or else active ingredients, for example active pharmaceutical ingredients or insecticides.

The volume fraction of functional additives (also referred to herein as functional particles or functional materials) in the constituting mixture has preferably been chosen such that it, at above 50%, comprises the main volume component in the web-spinning air-moist filaments/fibers. In one particular embodiment, in which the diameter of the functional particles is about ¼ of the average filament/fiber diameter of the air-moist spunbonded web, the individual particles in the filaments/fibers have points of contact and so the functional properties can develop in an advantageous manner.

The additive-containing fabrics comprising particle-containing filaments/fibers are produced via a spunbond process. A doped polymer-containing spinning solution is used in the fiberization process, the direct solvent preferably being an aprotic solvent. Useful direct solvents, especially for cellulose, include particularly N-methylmorpholine N-oxide or N-methylmorpholine N-oxide monohydrate, ionic liquids, such as 1-ethyl-3-methylimidazolium acetate, 3-ethyl-1-methylimidazolium chloride or 3-butyl-1-methylimidazolium chloride, dimethylformamide, dimethylacetamide or dimethyl sulfoxide mixed with lithium chloride or NaOH-thiourea-water or optionally mixtures thereof. The spinning solution with the functional particles and the dissolved polymer is extruded through die holes having a diameter of 0.1 to 1.1 mm and preferably of 0.3 to 0.7 mm.

The resulting strands are attenuated, immediately upon leaving the dies, by their own weight and/or an obliquely forwardly directed air stream, the intensity of which is adapted to the reduced thread-drawing capacity of the spun mixture due to the functional particles, within a short path, in the longitudinal direction, into filaments and fibers having aspect ratios above 1000, preferably above 5000 and more preferably above 40 000. Fiber cracks resulting in the process do not lead to discontinuation of the process and have no adverse effect on the step of web production. The effect they do have is that fibers of varying length and varying diameter are present in a web. The fibers are subsequently (in the course of transitioning into the tensionless space) stabilized in their shape before the onset of longitudinal relaxation. This is accomplished by transforming the dissolved state of the polymer into an at least partially undissolved state, either by evaporating the solvent in the temperature-controlled air stream or by means of a stream of fine droplets, especially of water or an aprotic liquid, by gelation and possible exchange of the solvent. On reaching a tack-free state, the fibers/filaments are laid down on a foraminous belt or drum to form a web, which can also be layered, and compacted by aspiration. The excess water enriched with solvent is separated off, the remaining solvent is rinsed out by repeated washing and subsequently the web obtained can be dried if desired, in which case the functional particles in the web-constituting filaments and/or fibers become, due to the deswelling of the polymer taking place in the process, mutually touching and joining to an increased extent in a manner which is property determining.

The directly dissolved polymer which binds the particles is a nonmelting polymer, i.e., a polymer where the softening point is above the decomposition point. It is preferably a representative from the group of natural polymers, for example from the group of polysaccharides, and more preferably cellulose, of the polysaccharide derivatives and of the proteins or protein derivatives, and/or from the group of solvent-formable synthetic polymers, for example polyacrylonitrile, polyvinyl alcohol, polyethylene oxide, polysulfone, meta-aramid or their copolymers.

The wet spunbonded web thus obtained can be subjected to textile processes (needling, water jet consolidation, chemical bonding) to consolidate, refine and form it, in which case the consolidating and refining of the web can take place before or after drying. An aftertreatment by, for example, coating, impregnating or with active ionic ingredients can follow.

A spunbonded web is a web of fibers and filaments which is randomly laid down directly after extrusion, and two or more plies can also be laid on top of one another. The mixture of fibers and filaments is the result of fiber breakages below the die which result from the high particle content but which do not lead to any interruption of the process. Moreover, the spunbonded web of the process according to the present invention does not consist of just fibers differing in length, but the fibers themselves have different thicknesses along their lengths or between each other. Fiber thickness is determined by various factors, such as the concentration of the solution, the blasting rate, the type of polymer, the particle size, and also the interaction of additives with other components of the solution and additive content. At laydown, intertwined and entangled fibers and filaments are produced and produce a firmly interbonded web. The high particle content, the particle size and the fiber breakages result in characteristic nonuniformities in the average diameter of the fibers, which are very readily visible under a microscope.

The advantage of producing the fabric from solutions of polymers versus production from polymer melts is that the particle content can be immeasurably higher, since solvent is present in the starting solution alongside polymer and additive and is then removed at a later time. The forces of cohesion are sufficient to ensure that a breakage will only occur rarely, while at the same time the network of the dissolved polymer retains its glidability in order that the particles may glide past one another at extrusion and stretching. In addition, various gel states of the fibers and filaments due to the exchange of the solvent for water can be utilized for further processing.

The high-functionality spunbonded web has a weight per unit area of 2 to 1000 g/m² and preferably of 5 to 500 g/m² and a thickness of 0.01 to 20 mm and preferably of 0.05 to 5 mm. It possesses additional incorporated and permanent functions, for example electrically conductive, absorbing, ion exchanging, antibacterial, temperature regulating, flame retardant, abrasive, reconditioning or combinations thereof.

In one particular embodiment, pore-forming agents which are particulate, for example Glauber's salt, can be integrated into the polymer solution. In the spunbonded web produced, the pore-forming agents then lead, during the washing process, to a spunbonded web of high-porosity fibers and filaments which, compared with sheetlike sponges, have a very much higher surface area.

The use of a high-functionality spunbonded web comprising particle-containing filaments/fibers ranges from apparel textiles, for example interlinings which store heat or deliver active ingredients, to industrial textiles with high functional benefit for various application sectors—depending on the type of functional particles, for example for hygiene applications, as wound dressings, as carrier materials for active ingredients or as carrier materials in composites, as building and transportation material, as cosmetic material or as filters, for example for the filtration and binding of air and water ingredients such as phosphates, nitrates and ammonium-nitrogen compounds. Owing to the special manifestation of functional properties, due to the high concentration of additives, these fibrous nonwoven webs are also suitable for layered composites with other fabrics. This can be accomplished by producing the high-functionality spunbonded web on another, previously laid fabric during spunbonded web production.

The examples which follow serve to illustrate the invention. Percentages therein are by weight, unless otherwise stated or immediately apparent from the context.

### Example 1 (Comparator)

A 0.1 kg quantity of a ground ion exchange resin (strong basic anion exchanger) having a particle diameter of D₉₉=14.8 μm was dispersed in 1.5 kg of a 9% cellulose solution in N-methylmorpholine N-oxide monohydrate (NMMO monohydrate) followed by homogenizing at 90° C. for 30 minutes. The spinning solution was subsequently gear pumped at 95° C. to a spinneret die (1200 holes having a diameter of 0.3 mm) and spun. However, consistent forming via an air gap (1=10 mm) was not possible because the emerging jets of solution became coalesced at the spinneret exit. Some of the fiber tow pieces formed were completely freed of solvent and, as far as possible, cut to a staple length of 40 mm, with the coalesced regions described being screened out as far as possible. The fibers were treated with a 1% sodium chloride solution and dried at 55° C. to constant weight. Secondary spinning into yarn was not possible. Web production was only marginally possible with a large number of short fibers and extreme truncation of the fibers being observed. The irregular looser portions of web were not further processable/utilizable. Consolidation by needling for stabilization was not possible since the web became completely destroyed in the process and disintegrated.

### Example 2

A cellulose solution produced as per Example 1 was solidified via melt-blow spinning process (solution blowing) at a solution temperature of 95° C., a blast with warm air at 80° C. and spraying with a water mist immediately on exit from the die blow unit and formed into a direct web by laydown on a foraminous belt. The forming operation was stable and the nonwoven obtained was, following complete extraction of the solvent and drying at 60° C., readily usable as ion exchange web. The functional web was mechanically so stable that it could be cut to size and introduced into the water treatment rig. An additionally performed moderate needling and thus further compaction was likewise possible without the web becoming destroyed in the process.

1. A high-functionality spunbonded web comprising fibers based on nonmeltable polymers containing one or more functional additives, wherein the fibers are intertwined and entangled, have a differing lengths, have aspect ratios above 1000 and form a firmly interbonded web, the fibers have an average diameter of 0.1 to 500 micrometers and also diameter fluctuations of at least 30% within and between fibers and the fibers in addition to the nonmeltable polymer contain more than 40 wt %, based on total fiber weight, of functional additives in solid and/or liquid form that are finely distributed within the fibers.

2. The spunbonded web as claimed in claim 1, wherein the functional additives are lipophilic substances.

3. The spunbonded web as claimed in claim 1, wherein the nonmeltable polymer binding the functional additives is a natural polymer and/or is a solution-formable synthetic polymer.

4. The spunbonded web as claimed in claim 1, wherein said web has a layered construction built from intertwined mechanically bonded filaments/fibers.

5. The spunbonded web as claimed in claim 1, wherein said web has a weight per unit area of 2 to 1000 g/m².

6. The spunbonded web as claimed in claim 1, wherein said web has a thickness of 0.1 to 20.

7. The spunbonded web as claimed in claim 1, wherein the fraction of functional additives is up to 96 wt %, based on total fiber weight.

8. A process for producing a high-functionality spunbonded web as claimed in claim 1, comprising
extruding a spinning solution comprising one or more functional additives, solvent and dissolved polymer through a spinneret die, the die having holes with a diameter of 0.1 to 1.5 mm, to form polymeric strands;
drawing the resulting polymeric strands into filaments and/or fibers, said drawing commencing immediately upon leaving the die, said drawing effected by the filaments and/or fibers own weight and/or an obliquely downwardly directed blasting stream the intensity of which is adapted to the reduced thread-drawing capacity of the spun mixture due to the functional additives, said drawing performed within a short path, in the longitudinal direction,
subsequently stabilizing the drawn filaments and/or fibers upon transitioning into a tensionless space, even before the onset of longitudinal relaxation, said filaments and/or fibers stabilized in their shape via a stream of temperature-controlled air and/or fine water droplets by consolidation/gelation and partial replacement of the solvent with water, wherein in spatial terms the stabilization can take place more or less offset to the die exit and gel-state fibers are obtained,
forming a web by laying down the stabilized fiber on a foraminous belt or drum, rinsing out the remaining solvent by repeated washing and optionally drying the web.

9. The process as claimed in claim 8, wherein the solvent comprises an aprotic solvent.

10. The process as claimed in claim 8, wherein the high-functionality spunbonded web is further consolidated, refined and formed by textile processes, and the consolidating and refining of the web is effected before or after drying.

11. The process as claimed in claim 8, wherein the high-functionality spunbonded web is stabilized by needling or water jet consolidation and/or additionally subjected to chemical crosslinking.

12. Apparel textiles, or industrial textiles comprising a spunbonded web as claimed in claim 1.

13. The spunbonded web as claimed in claim 2, wherein the functional additives are activated carbon, superabsorbents, ion exchange resins, piezoelectric materials, phase change materials, metal oxides, flame retardants, abrasives, zeolites, sheet-silicates, modified sheet-silicates and/or cosmetics.

14. The spunbonded web as claimed in claim 2, wherein the functional additives are paraffins.

15. The spunbonded web as claimed in claim 3, wherein the natural polymer is a polysaccharide, a polysaccharide derivative and/or a protein or protein derivative and the synthetic polymer is polyacrylonitrile or a copolymer with acrylonitrile units, polyvinyl alcohol, polyethylene oxide, polysulfone and/or meta-aramid.

16. The spunbonded web as claimed in claim 3, wherein the natural polymer is a cellulose.

17. The spunbonded web as claimed in claim 5, wherein said web has a weight per unit area of 5 to 500 g/m².

18. The spunbonded web as claimed in claim 6, wherein said web has a thickness of 0.5 to 5 mm.

19. A process for producing a high-functionality spunbonded web as claimed in claim 8, wherein the die holes have a diameter of 0.3 to 0.7 mm.

20. Apparel textiles or industrial textiles as claimed in claim 12, wherein the apparel textiles are interlinings, and the industrial textiles are hygiene textiles, wound dressings, carrier materials for active ingredients, carrier materials for composites, building material, transportation material, cosmetic material or filters.
